(19) United States
(12) Patent Application Publication
(10) Pub. No.: US 2025/0115584 A1
(43) Pub. Date: Apr. 10, 2025

(54) High-Purity Losartan Potassium and Preparation Method Therefor

(71) Applicant: ZHEJIANG HUAHAI PHARMACEUTICAL CO., LTD., Zhejiang (CN)

(72) Inventors: Shengjun Li, Zhejiang (CN); Yang Zheng, Zhejiang (CN); Aixing Li, Zhejiang (CN); Cuiting Liang, Zhejiang (CN); Mingyong Bai, Zhejiang (CN); Zhantao Zhang, Zhejiang (CN); Zunjun Liang, Zhejiang (CN); Fengfeng Yan, Zhejiang (CN)

(21) Appl. No.: 18/722,985

(22) PCT Filed: Dec. 14, 2022
(86) PCT No.: PCT/CN2022/138848

§ 371 Date: Jun. 21, 2024

(30) Foreign Application Priority Data
Dec. 22, 2021 (CN) 202111577590.0

(51) Int. Cl. C07D 403/10; A61K 31/4178

(52) U.S. Cl. CPC C07D 403/10; A61K 31/4178

(57) ABSTRACT

The present invention belongs to the field of pharmaceutical and chemical engineering, and relates to a high-purity losartan potassium and a preparation method therefor. The present invention also relates to a high-purity losartan or a pharmaceutically acceptable salt thereof and a preparation method therefor. The present invention uses organic phosphorus to reduce an azide compound to an amino compound so that the genotoxicity of losartan or a pharmaceutically acceptable salt thereof can be well controlled. The method of the present invention involves fewer steps, uses a small amount of reagent, does not require activated carbon decolorization, and produces a high-purity product, and therefore is suitable for industrial production and is very practical.

The present application claims the priority of the Chinese Patent Application No. 202111577590.0, with the title of “HIGH-PURITY LOSARTAN POTASSIUM AND PREPARATION METHOD THEREFOR”, filed on Dec. 22, 2021 before the China National Intellectual Property Administration, which is incorporated herein by reference in its entirety.

### FIELD OF THE INVENTION

The present invention relates to a high-purity losartan potassium and preparation method therefor, and belongs to the field of pharmaceutical and chemical engineering.

### BACKGROUND OF THE INVENTION

Losartan potassium is an antihypertensive drug developed by Merck in the United States. It was first marketed in 1994 and it is also the first angiotensin II receptor antagonist for the treatment of hypertension worldwide. Blood pressure is reduced by blocking type I angiotensin II receptor and inhibiting vasoconstriction and the release of aldosterone. The structure is represented as follows:

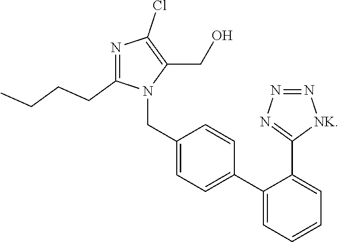

Losartan is an important intermediate for the synthesis of losartan potassium.

A common synthetic route of losartan potassium is as follows:

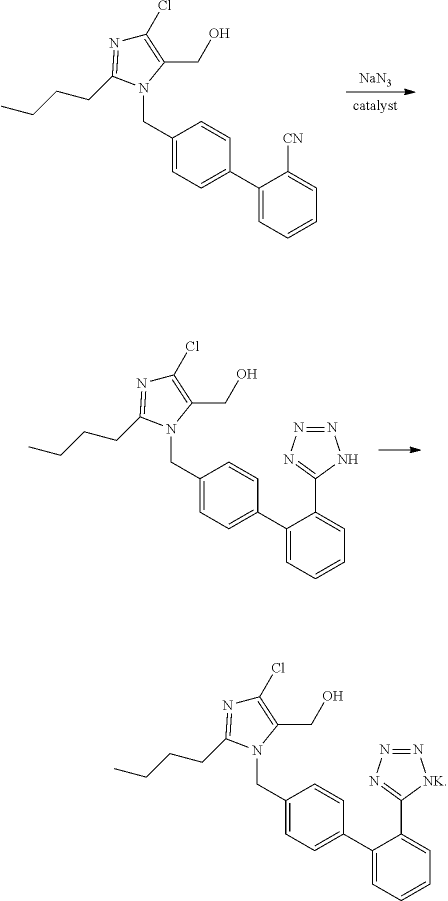

In the prior art, losartan is generally obtained by a cycloaddition reaction of compound 1 and sodium azide to construct tetrazole. Excessive sodium azide is used in the reaction process, which is easy to produce azide impurities (such as inorganic azide salt and organic azide impurities), which are difficult to remove in post-processing. Azide compounds may inhibit the activity of variety enzymes such as cytochrome oxidase, and lead to phosphorylation and abnormal cell respiration, result in extreme reduction of vascular tension; damage to biological cells, and hinder biological metabolism; and may directly cause damage of DNA even at lower concentrations, lead to mutagenesis of DNA, thus resulting in cancer. Therefore, it is pointed out in the guidelines of the International Council for Harmonization of Technical Requirements for Pharmaceuticals for Human Use (ICH)-M7 that azides are impurities that may cause genetic mutation, and their content in drugs and pharmaceutical intermediates must be strictly controlled in the process of drug production.

In addition, azide impurities is easy to affect the appearance of the product when present at a high content, and need to be decolorized in the reaction process.

### SUMMARY OF THE INVENTION

In one aspect, the present invention provides a preparation method of a losartan or a pharmaceutically acceptable salt thereof, comprising the following steps:

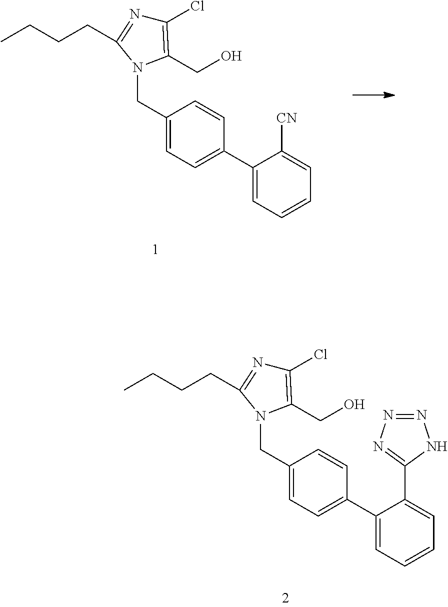 [SMILES: CCCCc1nc(Cl)c(CO)n1Cc1ccc(-c2ccccc2C#N)cc1 ; CCCCc1nc(Cl)c(CO)n1Cc1ccc(-c2ccccc2-c2nnn[nH]2)cc1]

(1) reacting Compound 1 with an azide reagent in the presence of an acidic reagent and a phase transfer catalyst in a water-insoluble organic solvent, and a crude product containing Compound 2 is obtained after the reaction is completed;
(2) treating the crude product obtained in step (1) with water and organic phosphine reagent.

In some embodiments of the present invention, the phase transfer catalyst is an ammonium salt phase transfer catalyst, preferably benzyltriethylammonium chloride, tetrabutylammonium bromide, tetrabutylammonium chloride or tetrabutylammonium hydrogen sulfate, and more preferably tetrabutylammonium bromide.

In some embodiments of the present invention, the molar ratio of the phase transfer catalyst to Compound 1 is 0.01:1 to 0.1:1, preferably 0.01:1 to 0.02:1, and more preferably 0.012:1 to 0.016:1.

In some embodiments of the present invention, the azide reagent is sodium azide.

In some embodiments of the present invention, the molar ratio of Compound 1 to the azide reagent is 1:1 to 1:2.3.

In some embodiments of the present invention, the molar ratio of Compound 1 to the azide reagent is 1:2.0 to 1:2.3.

In some embodiments of the present invention, the acidic reagent is triethylamine hydrochloride, and the molar ratio of Compound 1 to the acidic reagent is 1:2 to 1:3.

In some embodiments of the present invention, the water-insoluble organic solvent is toluene or xylene.

In some embodiments of the present invention, the organic phosphine reagent is a trivalent organophosphorus compound, preferably one or more of triphenylphosphine, tri-p-tolylphosphine, tri(2-furyl)phosphine and tri-tert-butylphosphine, further preferably triphenylphosphine.

In some embodiments of the present invention, the amount of the organic phosphine reagent is 0.1 mol. % to 2 mol. %, preferably 0.2 mol. % to 2 mol. %, more preferably 1.0 mol. % to 1.5 mol. %, and further more preferably 1.2 mol. % to 1.5 mol. %, relative to Compound 1.

In some embodiments of the present invention, in step (2), the organic phosphine reagent is added and the reaction is carried out at a reaction temperature of 20° C. to 70° C. for a reaction time of 0.5 h to 2 h.

In some embodiments of the present invention, in step (1), after completion of the reaction, the crude product containing Compound 2 is obtained by a process comprising the following steps: adding an alkaline solution and washing the mixture after the reaction is completed to divide the reaction system into three layers, and separating the material layer; preferably, the alkaline solution is sodium carbonate solution.

In some embodiments of the present invention, the amount of water added in step (2) is 0.5 to 3 times by volume of the water-insoluble organic solvent in step (1).

In some embodiments of the present invention, the amount of water added in step (2) is 0.7 to 1.2 times by volume of the water-insoluble organic solvent in step (1).

In some embodiments of the present invention, the preparation method of the present invention further comprises step (3):
(3) purifying the crude product obtained after the treatment in step (2) to obtain losartan.

In some embodiments of the present invention, the purifying process comprises cooling, acidifying, heating and crystallizing the crude product to obtain losartan.

In some embodiments of the present invention, in step (3), the cooling temperature is 0° C. to 10° C.; the pH of acidifying is 2 to 6; and the heating temperature is 20° C. to 25° C.

In some embodiments of the present invention, the preparation method of the present invention further comprising step (4): forming salt of the losartan obtained in step (3) in a mixed solution of potassium hydroxide in isopropanol-water to obtain the finished product of losartan potassium.

In another aspect, the present invention provides a preparation method of losartan, comprising the following steps:

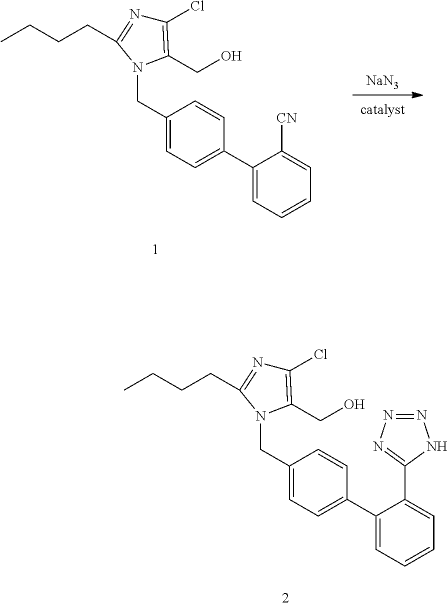 [SMILES: CCCCc1nc(Cl)c(CO)n1Cc1ccc(-c2ccccc2C#N)cc1 ; CCCCc1nc(Cl)c(CO)n1Cc1ccc(-c2ccccc2-c2nnn[nH]2)cc1 ; "NaN3"]

(1) reacting Compound 1 with sodium azide in the presence of triethylamine hydrochloride and tetrabutylammonium bromide in toluene, and a crude product containing Compound 2 is obtained after the reaction is completed;
(2) treating the crude product obtained in step (1) with water and triphenylphosphine;
(3) purifying the crude product to obtain losartan.

In some embodiments of the present invention, in step (1), the molar ratio of Compound 1 to triethylamine hydrochloride is 1:2 to 1:3; the molar ratio of the tetrabutylammonium bromide to Compound 1 is 0.01:1 to 0.1:1, preferably 0.01:1 to 0.02:1, and more preferably 0.012:1 to 0.016:1; and the molar ratio of Compound 1 to sodium azide is 1:1 to 1:2.3, preferably 1:2.0 to 1:2.3.

In step (2), the amount of water added is 0.7 to 1.2 times of toluene by volume; the molar ratio of the amount of triphenylphosphine to Compound 1 is 1:80 to 1:100; triphenylphosphine is added and the reaction is carried out at a reaction temperature of 20° C. to 70° C. for a reaction time is 0.5 h to 2 h.

In step (3), the purifying process comprises the steps of cooling, acidifying, heating, and crystallizing the system containing the crude product; preferably, the cooling temperature is 0° C. to 10° C.; the pH of acidifying is 2 to 6; and the heating temperature is 20° C. to 25° C.

In another aspect, the present invention provides a preparation method of losartan potassium, comprising the following step:
(4): forming salt of the losartan obtained by the preparation method of the present invention in a mixed solution of potassium hydroxide in isopropanol-water to obtain the finished product losartan potassium;

In another aspect, the present invention provides the losartan and/or losartan potassium prepared by the present invention, wherein the contents of Compounds I and II are less than 0.1%, preferably less than 10 ppm, more preferably less than 3 ppm, and
the structural formulae of Compounds I and II are as follows:

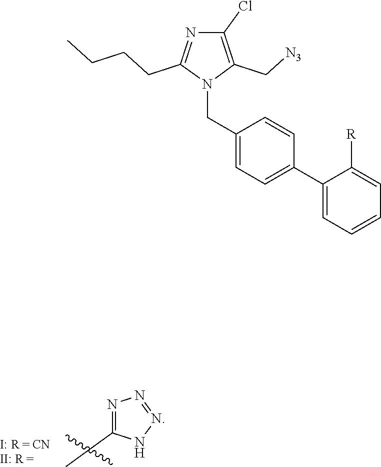

In another aspect, the present invention provides a pharmaceutical composition, comprising a therapeutically effective amount of losartan and/or losartan potassium of claim 19, and optionally one or more pharmaceutically acceptable carriers and/or diluents.

In the preparation method of losartan or pharmaceutically acceptable salt thereof of the present invention, the azide compound is converted into an amino compound, and thus the genotoxicity of the medicament is effectively controlled, such that the safety of the medicament is improved.

The contents of Compounds I and II in the losartan and/or losartan potassium prepared by the present invention are less than 0.1%, preferably less than 10 ppm, and more preferably less than 3 ppm. The structural formulae of Compounds I and II are as follows:

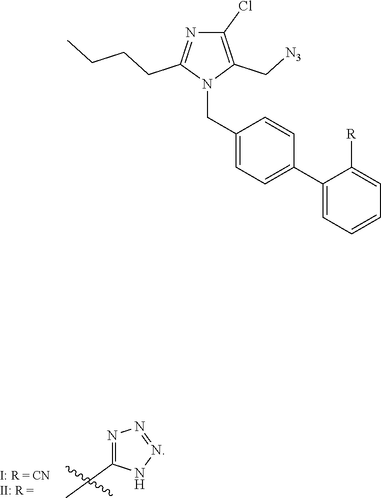

The method of the present invention involves fewer steps, less reagent consumption, and no need for decolorization by activated carbon, and the product has a high purity, a lower cost, and is therefore suitable for industrial production.

The present invention further provides a pharmaceutical composition, comprising a therapeutically effective amount of losartan and/or losartan potassium prepared by the present invention, and optionally one or more pharmaceutically acceptable carriers and/or diluents.

### BRIEF DESCRIPTION OF THE DRAWINGS

FIG. 1 is a mass spectrum of 4′-((5-(azidomethyl)-2-butyl-4-chloro-1H-imidazol-1-yl) methyl)-[1,1′-biphenyl]-2-nitrile (Compound I) prepared in Example 1.

FIG. 2 is a H¹-NMR spectrum of 4′-((5-(azidomethyl)-2-butyl-4-chloro-1H-imidazol-1-yl) methyl)-[1,1′-biphenyl]-2-nitrile (Compound I) prepared in Example 1.

FIG. 3 is a mass spectrum of 4′-((5-(aminomethyl)-2-butyl-4-chloro-1H-imidazol-1-yl) methyl)-[1,1′-biphenyl]-2-nitrile (Compound III) prepared in Example 3.

FIG. 4 is a H¹-NMR spectrum of 4′-((5-(aminomethyl)-2-butyl-4-chloro-1H-imidazol-1-yl)methyl)-[1,1′-biphenyl]-2-nitrile (Compound III) prepared in Example 3.

FIG. 5 is a LCMS-MS detection spectrum of Compound I.

FIG. 6 is a LCMS-MS detection spectrum of Compound I in losartan potassium prepared in Example 5.

FIG. 7 is a LCMS-MS detection spectrum of Compound II.

FIG. 8 is a LCMS-MS detection spectrum of Compound II in losartan potassium prepared in Example 5.

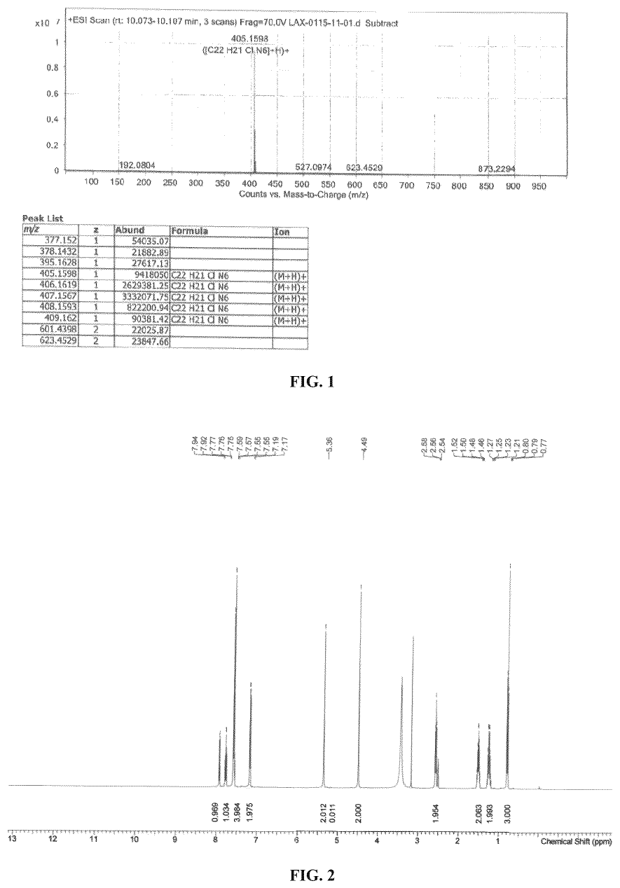

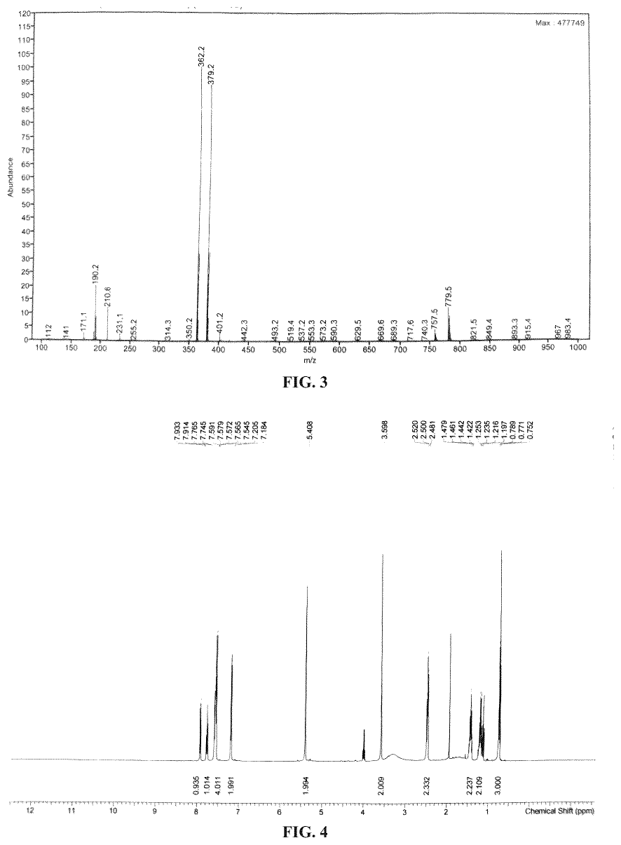

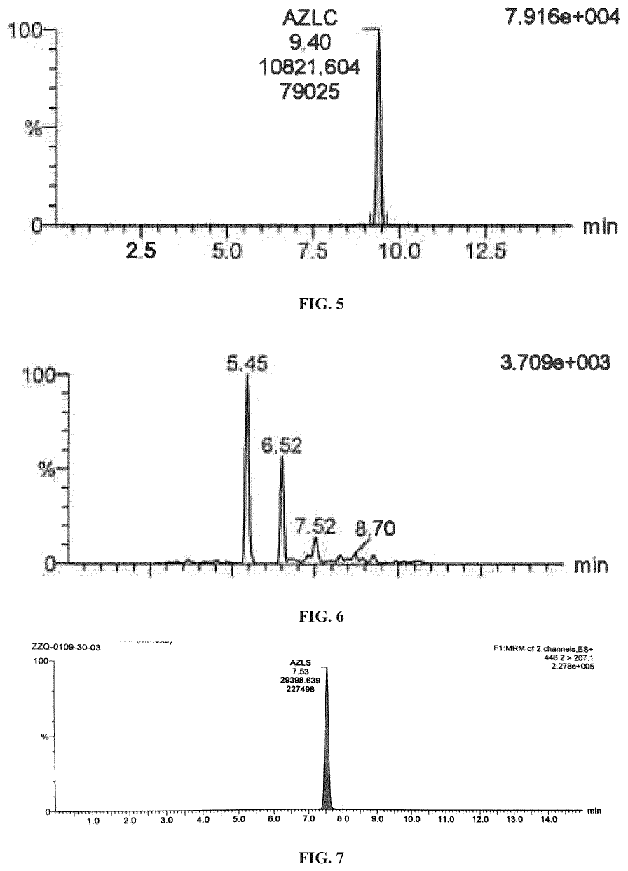

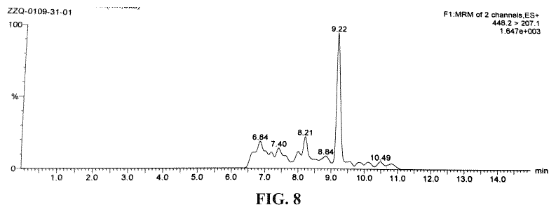

### DETAILED DESCRIPTION

The following specific embodiments are further detailed descriptions of the present invention.

In the present invention, unless otherwise stated, the scientific and technical terms used in the present invention have the meanings commonly understood by the skilled in the art. Moreover, the laboratory operation steps used in the present invention are all routine steps widely used in the corresponding fields.

### Definitions and Explanations

As used herein, the terms “having”, “comprising”, “containing” and “including” are to be interpreted as open-ended, indicating the presence of enumerated elements but not excluding the presence, appearance, or addition of any other one or more elements not enumerated.

All ranges recited herein include those endpoints reciting the range between the two values. Whether indicated or not, all values recited herein include the degree of expected experimental, technical and instrumental errors of the given technique used to measure the values.

In the present invention, % is a weight/weight (w/w) percentage unless otherwise stated.

Unless otherwise stated, any numerical value, such as a concentration or concentration range described herein, should be understood to be modified in all instances by the term “about”.

In the present invention, unless otherwise stated, the term “about” is intended to define its modified numerical value, indicating that such value can vary within a certain range. When there is no stated range (e.g., the error range or the standard deviation of the mean value given in a chart or datasheet), the term “about” should be understood to mean a larger range that includes the stated value, as well as a range that is rounded to that number considering significant figures, and a range that includes plus or minus 5% of the stated value.

The term “water-insoluble organic solvent” used in the present invention refers to organic solvents that are not miscible with water, including but not limited to, aromatic hydrocarbon organic solvents (such as toluene, xylene), aliphatic hydrocarbon organic solvents (such as petroleum ether, n-hexane), halogenated hydrocarbon organic solvents (such as dichloromethane, dichloroethane), ester organic solvents (such as ethyl acetate, butyl acetate), and ether organic solvents (such as diethyl ether, tetrahydrofuran).

In some embodiments of the present invention, the water-insoluble organic solvent is selected from one or more of toluene, xylene, ethyl acetate, butyl acetate, dichloromethane, and tetrahydrofuran.

In some embodiments of the present invention, the water-insoluble organic solvent is toluene or xylene.

In some embodiments of the present invention, the volume-mass ratio of the water-insoluble organic solvent to Compound 1 is 1 mL/g to 10 mL/g, such as 1 mL/g, 2 mL/g, 3 mL/g, 4 mL/g, 5 mL/g, 6 mL/g, 7 mL/g, 8 mL/g, 9 mL/g, 10 mL/g or any values and ranges there between.

In some embodiments of the present invention, the volume-mass ratio of the water-insoluble organic solvent to Compound 1 is 3 mL/g to 4 mL/g, such as 3.0 mL/g, 3.1 mL/g, 3.2 mL/g, 3.3 mL/g, 3.4 mL/g, 3.5 mL/g, 3.6 mL/g, 3.7 mL/g, 3.8 mL/g, 3.9 mL/g, 4.0 mL/g or any values and ranges there between.

The term “acidic reagent” used herein means proton donors, such as Lewis acid, a strong acid salt of a weak base, or a mixed system of weak base and strong acid. Lewis acid can be zinc chloride or lithium chloride, preferably zinc chloride; the strong acid salt of a weak base can be triethylamine hydrochloride, pyridine hydrochloride, triethylamine sulfate, pyridine sulfate, preferably triethylamine hydrochloride. In some embodiments of the present invention, in the mixed system of weak base and strong acid, the weak base is triethylamine, and the strong acid is hydrochloric acid or sulfuric acid.

In some embodiments of the present invention, the mixed system of weak base and strong acid is a mixed system of triethylamine and concentrated sulfuric acid, wherein the molar ratio of triethylamine to sulfuric acid may be 2:1.

In some embodiments of the present invention, the mixed system of weak base and strong acid is a mixed system of triethylamine and concentrated hydrochloric acid, wherein the molar ratio of triethylamine to hydrochloric acid may be 1:1.

In some embodiments of the present invention, the acidic reagent is triethylamine hydrochloride, the molar ratio of Compound 1 to the acidic reagent is 1:2 to 1:3.

In some embodiments of the present invention, the molar ratio of Compound 1 to triethylamine hydrochloride is 1:2 to 1:3, such as 1:2.0, 1:2.1, 1:2.2, 1:2.25, 1:2.26, 1:2.27, 1:2.28, 1:2.3, 1:2.4, 1:2.5 or 1:3.

The term “phase transfer catalyst” used in the present invention refers to a type of catalyst that can help the reactants transferring from one phase to another phase where the reaction can occur, thereby accelerating the reaction rate of the heterogeneous system. The phase transfer catalyst in the present invention can be an ammonium salt phase transfer catalyst, preferably benzyltriethylammonium chloride, tetrabutylammonium bromide, tetrabutylammonium chloride or tetrabutylammonium hydrogen sulfate, and more preferably tetrabutylammonium bromide.

In some embodiments of the present invention, the molar ratio of the phase transfer catalyst to Compound 1 is 0.01:1 to 0.1:1, preferably 0.01:1 to 0.02:1, and more preferably 0.012:1 to 0.016:1, such as 0.012:1, 0.013:1, 0.014:1, 0.015:1, or 0.016:1, or any values and ranges there between.

In some embodiments of the present invention, the phase transfer catalyst is tetrabutylammonium bromide, and the molar ratio of tetrabutylammonium bromide to Compound 1 is 0.01:1 to 0.1:1, preferably 0.01:1 to 0.02:1, and more preferably 0.012:1 to 0.016:1, such as 0.012:1, 0.013:1, 0.014:1, 0.015:1, or 0.016:1, or any values and ranges there between.

In the present invention, the term “crude product containing Compound 2” refers to a composition containing Compound 2, which comprises any other substances except Compound 2. The crude product containing Compound 2 can be, for example, a solution containing Compound 2, a mixture containing Compound 2 upon concentration, or a material layer containing the compound. Other substances in the composition include, but are not limited to, solvent, inorganic azide compounds, or organic azide compounds.

In some embodiments of the present invention, after the reaction in step (1) is completed, an alkaline solution is added to wash the mixture, such that the reaction system is divided into three layers, and a material layer is separated out. The crude product containing Compound 2 is the material layer separated above.

The term “azide reagent” used in the present invention refers to a precursor that can provide an azide group, such as sodium azide (NaN₃), potassium azide (KN₃), lithium azide (LiN₃), trimethylsilyl azide (TMSA), diphenylphosphoryl azide (DPPA), tributyltinyl azide (TBSnA), ethyl azidoacetate (AAE), tetrabutylammonium azide (TBAA), and the like, preferably sodium azide. In the present invention, the reaction between Compound 1 and the azide reagent is a cycloaddition reaction which constructs a tetrazole ring. In general, a large excess of the azide reagent relative to Compound 1 is required in this reaction. For example, in CN110467604B, the molar ratio of sodium azide to Compound 1 is 2.5 to 3.5. In the present invention, after adding the phase transfer catalyst, the reaction efficiency of Compound 1 and azide reagent is improved, the utilization rate of azide reagent is improved, and the amount of azide reagent needed is reduced. In the present invention, the molar ratio of Compound 1 to azide reagent is 1:1 to 1:2.3, such as 1:1, 1:1.5, 1:2.0, 1:2.3 or any values and ranges there between; preferably 1:2.0 to 1:2.3, such as 1:2.0, 1:2.1, 1:2.2, 1:2.3, or any values and ranges there between. After the reaction is complete, the level of azide-related impurities produced in the reaction system is lower, and there are fewer impurities in the system to affect the color of the product.

In the present invention, the azide-related impurities produced after the reaction of step (1) is completed may be inorganic azide compounds or organic azide compounds.

The terms “organic azide compound” and “organic azide” used in the present invention have the same meaning and refer to organic compounds containing an azide group.

In some embodiments of the present invention, the organic azide compound is an organic azide compound impurity produced during the synthesis of losartan or a pharmaceutically acceptable salt thereof. These organic azides are difficult to remove in post-processing, seriously affecting the quality and color of losartan or a pharmaceutically acceptable salt thereof such as losartan potassium. Further, azide compounds belong to possible genetic mutagenic impurities in the (ICH)-M7 guidelines, and the content thereof in drugs and pharmaceutical intermediates needs to be strictly controlled.

In some embodiments of the present invention, the organic azide compound is an organic azide compound impurity described in IN202111034277A.

In some embodiments of the present invention, the organic azide compounds are Compound I and Compound II shown in the following formulae,

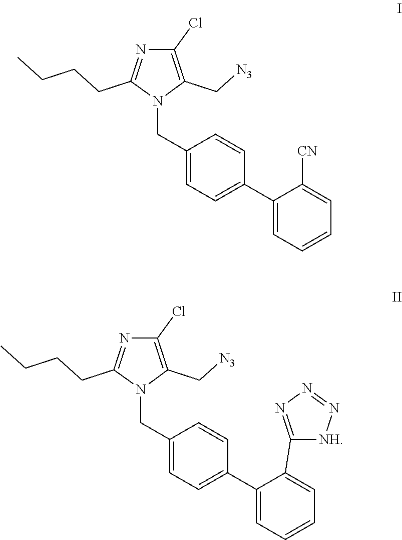 [SMILES: CCCCc1nc(Cl)c(CN=[N+]=[N-])n1Cc1ccc(-c2ccccc2C#N)cc1 ; CCCCc1nc(Cl)c(CN=[N+]=[N-])n1Cc1ccc(-c2ccccc2-c2nnn[nH]2)cc1]

In some embodiments of the present invention, treating the crude product obtained in step (1) with water and an organic phosphine reagent can remove residual organic azides in the system.

In some embodiments of the present invention, water and triphenylphosphine are added after the reaction is completed, reducing the azide group of the organic azide compound impurity to an amine group to obtain an organic amine compound impurity. Organic amine compound impurity is not a genotoxic impurity and only need to be controlled by the standards of impurities related to general drugs.

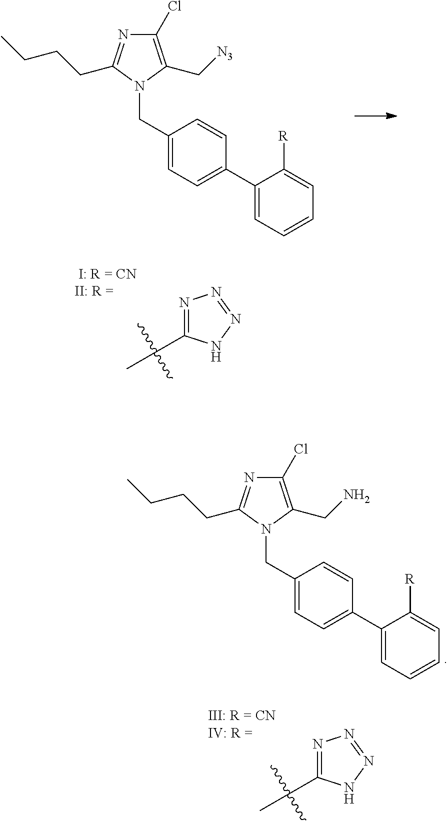

In the present invention, the reaction of the organic azide compound and the organic phosphine reagent produces an iminophosphorane compound, and hydrolysis thereof produces amine compounds. In the present invention, water and organic phosphine reagent are added in an amount that is able to convert the organic azide compound in the system of the present invention into an amine compound.

In some embodiments of the present invention, the amount of water added is 0.5 to 3 times by volume of the water-insoluble organic solvent, such as 0.5 times, 0.6 times, 0.7 times, 0.8 times, 0.9 times, 1.0 times, 1.2 times, 1.4 times, 1.6 times, 1.8 times, 1.9 times, 2.0 times or any ranges there between.

In some embodiments of the present invention, the amount of water added is 0.7 to 1.2 times by volume of the water-insoluble organic solvent, such as 0.7 times, 0.8 times, 0.9 times, 1.0 times, 1.1 times, 1.2 times or any values or ranges there between.

In some embodiments of the present invention, a hydrophilic organic solvent can also be added; preferably the hydrophilic organic solvent is selected from one or more of tetrahydrofuran, acetone, methanol, and acetonitrile; preferably, the content of water in the material layer is greater than or equal to 50%, and further preferably, the content of water in the material layer is greater than or equal to 90%.

In the present invention, the organic phosphine reagent is a trivalent organophosphorus compound.

In some embodiments of the present invention, the organic phosphine reagent is one or more of triphenylphosphine, tri-p-tolylphosphine, tri(2-furyl)phosphine and tri-tert-butyl phosphine.

In some embodiments of the present invention, the organic phosphine reagent is triphenylphosphine.

In some embodiments of the present invention, the amount of the organic phosphine reagent is 0.1% to 2% relative to Compound 1 by molar ratio. In some embodiments of the present invention, the amount of the organic phosphine reagent is 0.2% to 2% relative to Compound 1 by molar ratio. In some embodiments of the present invention, the amount of the organic phosphine reagent is 1.0% to 1.5% relative to Compound 1 by molar ratio. In some embodiments of the present invention, the amount of the organic phosphine reagent is 1.2% to 1.5% relative to Compound 1 by molar ratio, such as 1.2%, 1.3%, 1,4%, 1.5% or any values or ranges there between.

In the present invention, less impurities are produced, and thus there is no need for decolorization. In the prior art, decolorization usually requires the addition of a decolorizing agent, such as activated carbon. Activated carbon has an adsorption effect, which may cause a loss of reaction reagents while adsorbing impurities, and may affect subsequent reactions.

In some embodiments of the present invention, in step (2), triphenylphosphine is added without adding activated carbon for decolorization.

In some embodiments of the present invention, in step (2), the organic phosphine reagent is added and the reaction is carried out at a reaction temperature of 20° C. to 70° C., preferably 40° C. to 60° C., such as 40° C., 45° C., 50° C., 55° C., 60° C. or any values or ranges there between, and the reaction time is 0.5 h to 2 h, such as 0.5 h, 1 h, 1.5 h, 2 h or any values or ranges there between.

The term “alkaline solution” in the present invention refers to an aqueous solution with a pH greater than 7, such as 8, 9, 10, 11, 12 or 13. Exemplary bases for preparing an “alkaline solution” include, but are not limited to, hydroxide salts (e.g., sodium hydroxide, potassium hydroxide, and lithium hydroxide), carbonates (e.g., sodium carbonate, potassium carbonate, lithium carbonate, and calcium carbonate) or bicarbonates (e.g., sodium bicarbonate, and potassium bicarbonate).

The known method disclosed in CN112679476A can be used as a preparation method of the compound of the present invention. For example, specifically, the compound of the present invention can be prepared in the following embodiments.

The washing step after the reaction is completed is as follows: adding a certain amount of alkaline solution to divide the reaction system into three layers after the reaction of Compound 1 with azide reagent is completed, wherein the upper layer is a water-insoluble organic solvent layer, the middle layer is a material layer, and the lower layer is a water layer; heating and stirring to dissolve azide ions in the water layer; and removing the water layer to basically completely remove the azide ions, and obtaining the intermediate material layer without loss of materials.

In some embodiments of the present invention, the mass concentration of the alkaline solution is 15% to 30%.

In some embodiments of the present invention, the mass concentration of the alkaline solution is 16% to 22%.

In some embodiments of the present invention, the volume amount of water is 2 to 5 times of the water-insoluble organic solvent.

In some embodiments of the present invention, in the washing step, the temperature of heating and stirring is 50° C. to 90° C., such as 50° C. to 60° C., 60° C. to 70° C., 70° C. to 80° C., 80° C. to 90° C. or any temperature ranges there between.

In some embodiments of the present invention, the stirring time is 0.5 h to 4 h, preferably 0.5 h to 2 h.

The washing can be performed for one or more times.

In some embodiments of the present invention, washing is performed for 1 to 5 times, preferably 2 to 3 times.

In some embodiments of the present invention, the alkaline solution is sodium carbonate solution.

In some embodiments of the present invention, the alkaline solution is saturated sodium carbonate solution.

In some embodiments of the present invention, the washing step after the reaction is completed is as follows: adding saturated sodium carbonate solution to divide the reaction system into three layers, removing the lower water layer and upper organic solvent layer, separating the middle material layer, and repeating the above washing step for 3 times.

In the present invention, Compound I and II or Compound III and IV can be used as standard reference substances for inspection or analysis of losartan potassium-related substances.

Compound I or II in the present invention can be prepared by the following method:

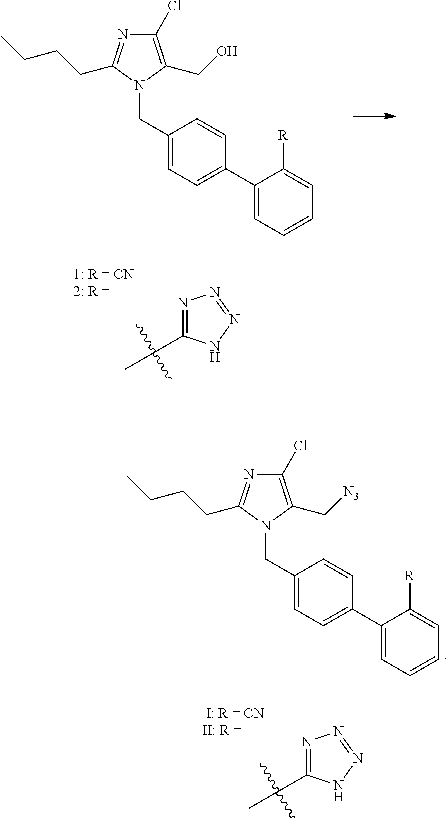

In a system of organic solvent and azide reagent under alkaline condition, Compound I is prepared by using Compound 1, or Compound II is prepared by using Compound 2.

Preferably, the organic solvent is an aprotic organic solvent, and further preferably, the organic solvent is one or more of toluene, xylene, ethyl acetate, butyl acetate, dichloromethane, and tetrahydrofuran.

Preferably, the base is an organic amine, preferably one or more of 1,8-diazabicycloundec-7-ene, triethylamine, N-methylmorpholine and pyridine.

Preferably, the azide reagent is one or more of diphenylphosphoryl azide, sodium azide, ethyl azidoacetate, azidotrimethylsilane, and tetrabutylammonium azide.

The present invention further provides a preparation method and use of any one of Compounds I to IV. Any one of Compounds I to IV can be used as a standard reference substance for inspection or analysis of losartan potassium-related substances, and can be used for the accurate location of the impurity in the analysis method of crude and finished products of losartan potassium and the quantitative study of the external standard method.

The detection conditions for the analysis method of crude and finished products of losartan potassium are as follows: mobile phase A: 0.01 mol/L potassium dihydrogen phosphate solution, with pH adjusted to 3.3 with concentrated phosphoric acid; mobile phase B: acetonitrile; column temperature: 25° C.; detection wavelength: 215 nm; flow rate: 1.0 mL/min; injection volume: 20 μL, gradient elution, preferably in the following elution gradient program:

| Time (min) | Mobile Phase A (% V/V) | Mobile Phase B (% V/V) |
|---|---|---|
| 0 | 62 | 38 |
| 10 | 62 | 38 |
| 35 | 20 | 80 |
| 45 | 20 | 80 |
| 46 | 62 | 38 |
| 55 | 62 | 38 |

The present application will be further described below in combination with specific examples. The examples are offered for more detailed description only, and will not limit the present invention in any form.

The HPLC analysis used in the present invention is as follows.

### 1. Chromatographic Conditions

Instrument: High Performance Liquid Chromatograph equipped with ultraviolet detector (UV)
Column: Shimpack CLC-ODS 150×6.0 mm, 5 μm
Mobile phase A: 0.01 mol/L potassium dihydrogen phosphate solution, pH adjusted to 3.3 with concentrated phosphoric acid
Mobile phase B: acetonitrile
Column temperature: 25° C.
Detection wavelength: 215 nm
Flow rate: 1.0 mL/min
Injection volume: 20 μL
Gradient Table:

| Time (min) | Mobile Phase A (% V/V) | Mobile Phase B (% V/V) |
|---|---|---|
| 0 | 62 | 38 |
| 10 | 62 | 38 |
| 35 | 20 | 80 |
| 45 | 20 | 80 |
| 46 | 62 | 38 |
| 55 | 62 | 38 |

### 2. Reagents

Acetonitrile: chromatographic purity
Concentrated phosphoric acid: analytically pure or chromatographically pure
Potassium dihydrogen phosphate: analytically pure or chromatographically pure
Water: ultra-pure water

### 3. Solution Preparation

Diluent: water:acetonitrile=65:35 (% V/V)
Blank solution: diluent
Test solution: 40 mg of the test article was accurately weighed into a 100 mL volumetric flask, and was dissolved with diluent by ultrasonic and diluted to the mark, and then the solution was mixed well.
Note: the test solution is stable within 35 h (stored at room temperature).

### 4. Steps

1 needle of blank solution and 1 needle of test solution were injected respectively, and the chromatographic processes were recorded.

### Preparation Route of Compounds I and II

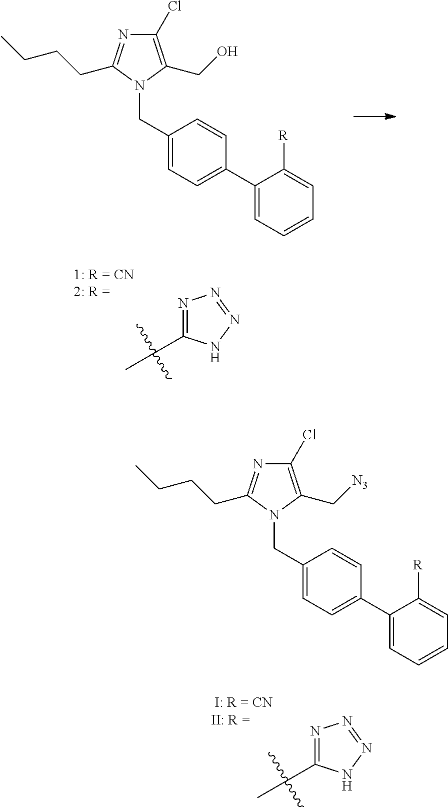

### EXAMPLE 1

### Preparation of Compound I

10 g of Compound 1 and 100 mL of toluene were sequentially added into a three-necked flask, and the mixture was cooled to 5° C. Then 10 g of diphenylphosphoryl azide was slowly added and stirred for 30 min while maintaining the temperature, and then 8 g of 1,8-diazabicycloundec-7-ene was slowly added and stirred while maintaining the temperature until the solid was completely dissolved. The solution was stirred further for 1 h while maintaining the temperature. Then the temperature of the reaction liquid was raised up to room temperature, and the solution was stirred further for 2 h to 3 h. Then the reaction was stopped, the reaction liquid was washed twice by water (50 mL*2), the organic phase was concentrated, and the concentrated liquid was separated by column chromatography to obtain a brown-yellow oily product I (developing agent: n-hexane:ethyl acetate=5:1), with a yield of 72% and an HPLC purity of greater than 96%.

### EXAMPLE 2

### Preparation of Compound II

10 g of Compound 2 (losartan) and 100 mL of toluene were sequentially added into a three-necked flask, and the mixture was cooled to 5° C. Then 12 g of diphenylphosphoryl azide was slowly added and stirred for 30 min while maintaining the temperature, and then 9 g of 1,8-diazabicycloundec-7-ene was slowly added and stirred while maintaining the temperature until the solid was completely dissolved. The solution was stirred further for 1 h. Then the temperature of the reaction liquid was raised up to room temperature, and the solution was stirred further for 2 h to 3 h. Then the reaction was stopped, the reaction liquid was washed twice by water (50 mL*2), the organic phase was concentrated, and the concentrated liquid was separated by column chromatography to obtain a pale yellow solid II (developing agent: n-hexane:ethyl acetate=5:1), with a yield of 75% and an HPLC purity of greater than 98%.

### Preparation Route of Compounds III and IV:

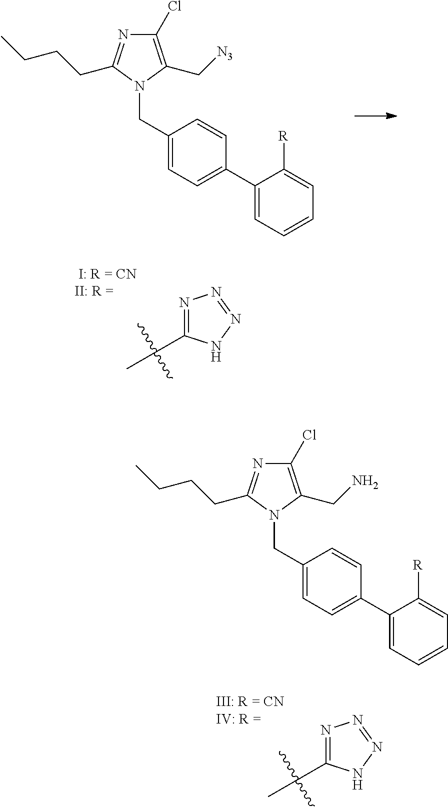

### EXAMPLE 3

### Preparation of Compound III

10 g of Compound I, 100 mL of tetrahydrofuran and 10 g of triphenylphosphine were sequentially added into a three-necked flask, and the mixture was raised up to 50° C. and reaction was performed for 5 h to 6 h while maintaining the temperature. Then 20 mL of water was added, and the temperature was maintained for 1 h to 2 h. After the reaction was completed, the reaction solution was concentrated to remove tetrahydrofuran, and then 100 mL of ethyl acetate was added into the concentrated solution and stirred until dissolved and clarified. 6 N hydrochloric acid was added slowly dropwise into the clarified solution to adjust pH to 1 to 3, and then layers separated, the aqueous phase was washed twice with ethyl acetate (50 mL*2). Then 100 mL of ethyl acetate was added into the washed aqueous phase, followed by an addition of 30% aqueous sodium hydroxide solution to adjust pH>11. Layers were separated, and the organic phase was washed with 50 mL of saturated brine once and concentrated to obtained a pale yellow solid III, with a yield of 68% and an HPLC purity of 95%.

### EXAMPLE 4

### Preparation of Compound IV

10 g of Compound II, 100 mL of tetrahydrofuran and 10 g of triphenylphosphine were sequentially added into a three-necked flask, and the mixture was raised up to 50° C. and reaction was performed for 5 h to 6 h while maintaining the temperature. Then 20 ml of water was added, the temperature was maintained for 1 h to 2 h. After the reaction was completed, the reaction solution was concentrated to remove tetrahydrofuran, and then 100 mL of ethyl acetate was added into the concentrated solution and stirred until dissolved and clarified. 6 N hydrochloric acid was added slowly dropwise into the clarified solution to adjust pH to 1 to 3, and then layers were separated, the aqueous phase was washed twice with ethyl acetate (50 mL*2). Then 100 mL of ethyl acetate was added into the washed aqueous phase, followed by an addition of 30% aqueous sodium hydroxide solution to adjust pH=4.5 to 5.5. Layers were separated, and the organic phase was washed with 50 mL of saturated brine once and concentrated to obtained a pale yellow solid IV, with a yield of 60% and an HPLC purity of 95%.

### EXAMPLE 5

### Preparation Method of High-Purity Losartan Potassium

200 mL of toluene, 47.6 g of triethylamine hydrochloride, 58.2 g of Compound 1, 0.8 g of tetrabutylammonium bromide (TBAB) and 21.6 g of sodium azide were sequentially added into a reaction flask. After the addition, the mixture was raised up to 100° C. to react for 48 h while maintaining the temperature. After the reaction was completed, the solution was washed for three times with 150 mL of saturated sodium carbonate solution, and the lower water layer and the upper toluene layer were separated out. 140 mL of water was added into the material layer, and the content of losartan-azide Compound I was detected to be 500 ppm, the content of Compound II was detected to be 2000 ppm, and the azido was detected to be less than 20 ppm. 0.5 g of triphenylphosphine was added into the solution and held at 45° C. to react for 1 h. The system was then cooled to 0° C. to 10° C., and 6 mol/L hydrochloric acid was added dropwise into the solution to adjust pH to 4. After acid adjustment, the temperature was raised to 20-25° C. and allowed to crystallize for 2 h. The mixture was suction filtered, and the solid was transformed to salt in a mixed solution of potassium hydroxide in isopropanol-water, which was then recrystallized, suction filtrated, and dried to obtain losartan potassium, with a yield of 75% and an HPLC purity of greater than 99.9%. The content of Compounds I and II in losartan potassium were detected to be below the detection limit (detection limit is 3.0 ppm).

### EXAMPLE 6

### Preparation Method of High-Purity Losartan Potassium

200 mL of toluene, 47.6 g of triethylamine hydrochloride, 58.2 g of Compound 1, 0.8 g of TBAB and 22.8 g of sodium azide were sequentially added into a reaction flask. After the addition, the mixture was raised up to 100° C. to react for 48 h while maintaining the temperature. After the reaction was completed, the solution was washed for three times with 150 mL of saturated sodium carbonate solution, and the lower water layer and the upper toluene layer were separated out. 140 mL of water was added into the material layer, and the content of losartan-azide Compound I was detected to be 500 ppm, the content of Compound II was detected to be 2000 ppm, and the azido was detected to be less than 50 ppm. 0.5 g of triphenylphosphine was added into the solution and held at 55° C. to react for 1 h. The system was then cooled to 0° C. to 10° C., and 6 mol/L hydrochloric acid was added dropwise into the solution to adjust pH to 4. After acid adjustment, the temperature was raised to 20-25° C. and allowed to crystallize for 2 h. The mixture was suction filtered, and the solid was transformed to salt in a mixed solution of potassium hydroxide in isopropanol-water, which was then recrystallized, suction filtrated, and dried to obtain losartan potassium, with a yield of 76% and an HPLC purity of greater than 99.9%. The content of Compounds I and II in losartan potassium were detected to be below the detection limit (detection limit is 3.0 ppm).

### EXAMPLE 7

### Preparation Method of High-Purity Losartan Potassium

200 mL of toluene, 47.6 g of triethylamine hydrochloride, 58.2 g of Compound 1, 0.8 g of TBAB and 21.6 g of sodium azide were sequentially added into a reaction flask. After the addition, the mixture was raised up to 102° C. to react for 45 h while maintaining the temperature. After the reaction was completed, the solution was washed for three times with 150 mL of saturated sodium carbonate solution, and the lower water layer and the upper toluene layer were separated out. 140 ml of water was added into the material layer, and the content of losartan-azide Compound I was detected to be 500 ppm, the content of Compound II was detected to be 2000 ppm, and the azido was detected to be less than 30 ppm. 1 g of triphenylphosphine was added into the solution and held at 60° C. to react for 2 h. The system was then cooled to 0° C. to 10° C., and 6 mol/L hydrochloric acid was added dropwise into the solution to adjust pH to 4. After acid adjustment, the temperature was raised to 20-25° C. and allowed to crystallize for 2 h. The mixture was suction filtered, and the solid was transformed to salt in a mixed solution of potassium hydroxide in isopropanol-water, which was then recrystallized, suction filtrated, and dried to obtain losartan potassium, with a yield of 76.5% and an HPLC purity of greater than 99.9%. The content of Compounds I and II in losartan potassium were detected to be below the detection limit (detection limit is 3.0 ppm).

1. A preparation method of a losartan or a pharmaceutically acceptable salt thereof, comprising the following steps: 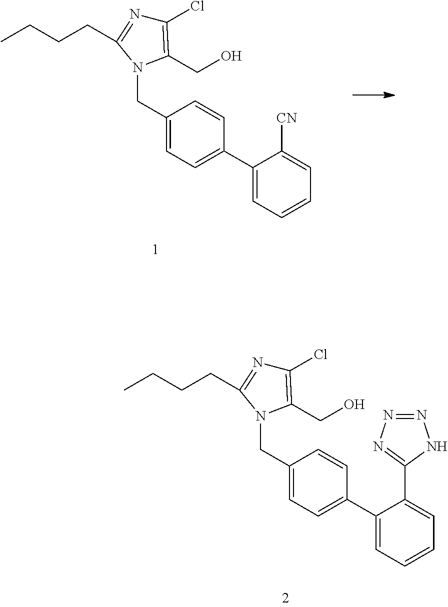 [SMILES: CCCCc1nc(Cl)c(CO)n1Cc1ccc(-c2ccccc2C#N)cc1 ; CCCCc1nc(Cl)c(CO)n1Cc1ccc(-c2ccccc2-c2nnn[nH]2)cc1]
(1) reacting a compound 1 with an azide reagent in a water-insoluble organic solvent in the presence of an acidic reagent and a phase transfer catalyst, and obtaining a crude product containing a compound 2 after the reaction is completed; and
(2) treating the crude product obtained in step (1) with water and an organic phosphine reagent.

2. The preparation method according to claim 1, wherein the phase transfer catalyst is an ammonium salt phase transfer catalyst.

3. The preparation method according to claim 1, wherein the molar ratio of the phase transfer catalyst to the compound 1 ranges from 0.01:1 to 0.1:1.

4. The preparation method according to claim 1, wherein the azide reagent is sodium azide; and/or
wherein the molar ratio of the compound 1 to the azide reagent ranges from 1:1 to 1:2.3.

5. (canceled)

6. The preparation method according to claim 1, wherein the acidic reagent is triethylamine hydrochloride, and the molar ratio of the compound 1 to the acidic reagent ranges from 1:2 to 1:3.

7. The preparation method according to claim 1, wherein the water-insoluble organic solvent is toluene or xylene.

8. The preparation method according to claim 7, wherein the organic phosphine reagent is a trivalent organophosphorus compound; and/or
wherein the amount of the organic phosphine reagent is 0.1 mol. % to 2 mol. % relative to the compound 1.

9. (canceled)

10. The preparation method according to claim 7, wherein, in step (2), the organic phosphine reagent is added and the reaction is carried out at a reaction temperature of 20° C. to 70° C. for a reaction time of 0.5 hour to 2 hours.

11. The preparation method according to claim 1, wherein in step (1), after completion of the reaction, the crude product containing the compound 2 is obtained by a process comprising the following steps: adding an alkaline solution and washing the mixture after the reaction is completed to divide the reaction system into three layers, and separating a material layer.

12. The preparation method according to claim 1, wherein the amount of water added in step (2) is 0.5 to 3 times by volume of the water-insoluble organic solvent in step (1).

13. The preparation method according to claim 1, further comprising a step (3):
(3) purifying the crude product obtained in step (2) to obtain losartan.

14. The preparation method according to claim 13, wherein in step (3), the purifying process comprises the steps of cooling, acidifying, heating, and crystallizing the crude product.

15. The preparation method according to claim 13, further comprising a step (4): forming a salt of the losartan obtained in step (3) in a mixed solution of potassium hydroxide in isopropanol-water to obtain the finished product of losartan potassium.

16. A preparation method of losartan potassium, comprising the following steps: 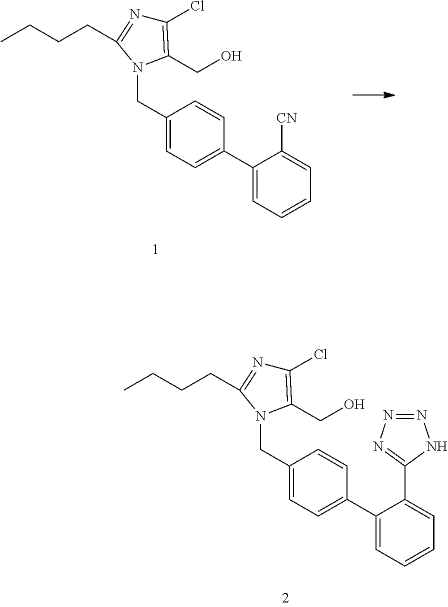 [SMILES: CCCCc1nc(Cl)c(CO)n1Cc1ccc(-c2ccccc2C#N)cc1 ; CCCCc1nc(Cl)c(CO)n1Cc1ccc(-c2ccccc2-c2nnn[nH]2)cc1]
(1) reacting a compound 1 with a sodium azide in toluene in the presence of triethylamine hydrochloride and tetrabutylammonium bromide, and a crude product containing a compound 2 is obtained after the reaction is completed;
(2) treating the crude product obtained in step (1) with water and triphenylphosphine; and
(3) purifying the crude product to obtain losartan.

17. The preparation method according to claim 16, wherein,
in step (1), the molar ratio of the compound 1 to triethylamine hydrochloride ranges from 1:2 to 1:3; the molar ratio of tetrabutylammonium bromide to the compound 1 ranges from 0.01:1 to 0.1:1; and the molar ratio of the compound 1 to sodium azide ranges from 1:1 to 1:2.3;
in step (2), the amount of water added is 0.7 to 1.2 times by volume of toluene; the molar ratio of the amount of triphenylphosphine to the compound 1 is 1:80 to 1:100; triphenylphosphine is added and the reaction is carried out at a reaction temperature of 20° C. to 70° C. and a reaction time of 0.5 hour to 2 hours; and
in step (3), the purifying process comprises the steps of cooling, acidifying, heating, and crystallizing the system containing the crude product.

18. A preparation method of losartan potassium, comprising the following step:
(4): forming a salt of losartan obtained by the preparation method according to claim 13 in a mixed solution of potassium hydroxide in isopropanol-water to obtain the finished product of losartan potassium.

19. Losartan, wherein the contents of compounds I and II are less than 0.1%, wherein
the structural formulae of compounds I and II are as follows:

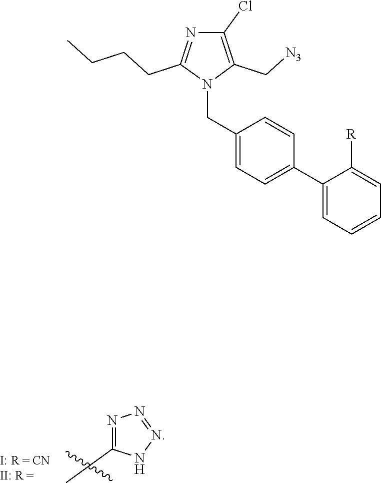

20. A pharmaceutical composition, comprising a therapeutically effective amount of losartan of claim 19, and optionally one or more pharmaceutically acceptable carriers and/or diluents.

21. Losartan potassium, wherein the contents of compounds I and II are less than 0.1%, wherein
the structural formulae of compounds I and II are as follows:

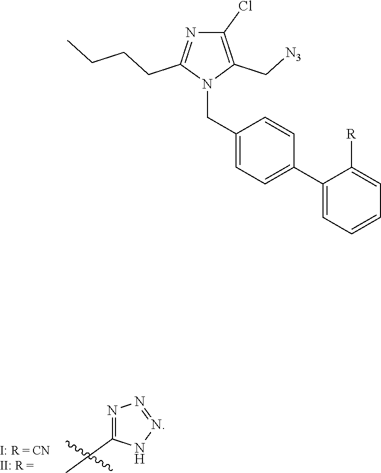

22. A pharmaceutical composition, comprising a therapeutically effective amount of losartan potassium of claim 21, and optionally one or more pharmaceutically acceptable carriers and/or diluents.
